(19) United States
(12) Patent Application Publication
(10) Pub. No.: US 2022/0401638 A1
(43) Pub. Date: Dec. 22, 2022

(54) PERCUTANEOUS CATHETER

(71) Applicant: TERUMO KABUSHIKI KAISHA, Tokyo (JP)

(72) Inventors: Kenji Yokoyama, Kanagawa (JP); Takehisa Mori, Kanagawa (JP)

(21) Appl. No.: 17/893,489

(22) Filed: Aug. 23, 2022

Related U.S. Application Data
(63) Continuation of application No. PCT/JP2021/006271, filed on Feb. 19, 2021.

(30) Foreign Application Priority Data
Mar. 6, 2020 (JP) 2020-038884

(51) Int. Cl. A61M 1/36; A61M 25/00; A61M 60/113; A61M 60/232; A61M 60/38; A61M 25/01

(52) U.S. Cl. CPC A61M 1/3659; A61M 25/007; A61M 60/113; A61M 60/232; A61M 60/38; A61M 25/0102

(57) ABSTRACT

A percutaneous catheter for blood removal includes a catheter tube extending in an axial direction with a plurality of distal side holes provided on a distal end part of the catheter tube and communicating a lumen of the catheter with an outside of the catheter tube. The plurality of distal side holes is spirally arranged in the axial direction of the catheter tube, and at least one distal side hole at the proximal end side of the spirally arranged side holes is formed to have a diameter smaller than a diameter of a distal side hole at the distal end side of the spirally arranged side holes.

### CROSS REFERENCE TO RELATED APPLICATIONS

This application is a continuation of PCT Application No. PCT/JP2021/006271, filed Feb. 19, 2021, based on and claiming priority to Japanese Application No. JP2020-038884, filed Mar. 6, 2020, both of which are incorporated herein by reference in their entirety.

### BACKGROUND OF THE INVENTION

The present invention relates to a percutaneous catheter.

Conventionally, treatment by percutaneous cardiopulmonary support (PCPS) has been performed in order to perform cardiopulmonary resuscitation, circulation assistance, and respiration assistance in emergency treatment. The percutaneous cardiopulmonary support is a method of temporarily assisting and substituting for a cardiopulmonary function using an extracorporeal circulation device.

The extracorporeal circulation device is provided with an extracorporeal circulation circuit formed of a centrifugal pump, an oxygenator, a blood removal path, a blood supply path and the like, and performs gas exchange on removed blood and supplies the blood to the blood supply path.

In a case where blood circulation is performed by the circulation circuit, the blood is circulated by a force of the pump driven by a motor. Therefore, in order to suitably perform the blood circulation, it is required to alleviate a pressure loss in a tube forming the circulation circuit.

In the extracorporeal circulation circuit, a catheter (cannula) provided with a lumen through which the blood flows is used for the blood removal path and the blood supply path.

In this regard, for example, JP 2011-83421A mentioned below discloses a blood removal catheter as a catheter used in the extracorporeal circulation circuit.

In the catheter disclosed in JP 2011-83421A, a plurality of blood removal holes is provided at the same position in a circumferential direction as seen in an axial direction. It is preferable to provide the plurality of blood removal holes at a distal end of the catheter in this manner from a viewpoint of efficiency. In the catheter disclosed in JP 2011-83421A, diameters of the plurality of blood removal holes provided in the axial direction are made substantially the same.

### SUMMARY OF THE INVENTION

The present inventors have found that, in a case where the plurality of blood removal holes is provided at the same position in the circumferential direction as seen in the axial direction, there is a possibility that blood removal efficiency is deteriorated due to collision of blood flowing in via the blood removal holes.

Moreover, in a case where the diameters of the plurality of blood removal holes provided in the axial direction are made substantially the same, a blood removal amount in the blood removal hole on a proximal end side is larger than the blood removal amount from the blood removal hole on a distal end side. In general, when the catheter is arranged in a living body, the blood removal hole on the distal end side out of the plurality of blood removal holes is arranged in the vicinity of the right atrium of the heart. Therefore, it is instead preferable that the blood removal amount in the blood removal hole on the distal end side is larger than the blood removal amount from the blood removal hole on the proximal end side.

The present invention is achieved in order to solve the above-described problem, and an object thereof is to provide a percutaneous catheter capable of suitably suppressing deterioration in blood removal efficiency even in a case where a plurality of side holes (blood removal holes) is provided, and making a blood removal amount in a side hole on a distal end side larger than a blood removal amount from a side hole on a proximal end side.

A percutaneous catheter for achieving the above-described object is a percutaneous catheter provided with a lumen through which blood passes. The percutaneous catheter includes a tube extending in an axial direction and side holes provided on a distal end part of the tube and communicating the lumen with the outside of the tube. A plurality of side holes is spirally arranged in the axial direction of the tube. Out of the side holes, a side hole on a proximal end side is formed to have a diameter smaller than that of the side hole on a distal end side.

More specifically, the percutaneous catheter may be provided with a lumen through which blood passes which is removed by the percutaneous catheter from a living body. The percutaneous catheter may include a tube extending in an axial direction between a distal end and a proximal end, and a distal tip. The tube includes a plurality of distal side holes provided on a distal end part of the tube and communicating the lumen with an outside of the tube configured to remove the blood from a first target in the living body. The plurality of distal side holes is spirally arranged in the axial direction of the tube along a virtual line from a proximal end side to a distal end side. One of the plurality of distal side holes at the proximal end side is formed to have a diameter smaller than a diameter of another one of the plurality of distal side holes at the distal end side. According to the percutaneous catheter formed as described above, since the side holes are formed spirally in the axial direction, it is possible to suitably suppress collision of blood flowing in via the side holes. Therefore, deterioration in blood removal efficiency may be suitably suppressed.

According to the percutaneous catheter formed in the above-described manner, since the side hole on the proximal end side is formed to have the diameter smaller than that of the side hole on the distal end side, a blood removal amount in the side hole on the distal end side may be made larger than the blood removal amount from the side hole on the proximal end side.

From above, according to the percutaneous catheter formed as described above, even in a case where a plurality of side holes is provided, it is possible to suitably suppress deterioration in blood removal efficiency and to make the blood removal amount in the side hole on the distal end side larger than the blood removal amount from the side hole on the proximal end side.

### BRIEF DESCRIPTION OF THE DRAWINGS

FIG. 1 is a system diagram illustrating an example of an extracorporeal circulation device to which a percutaneous catheter according to an embodiment of the present invention is applied.

FIG. 2 is a side view illustrating a state before a stylet is inserted into a catheter according to a first embodiment.

FIG. 3 is a side cross-sectional view illustrating the catheter according to the first embodiment.

FIG. 4 is a side view illustrating a state of the catheter of the first embodiment after the stylet is inserted into the catheter.

FIG. 5A is a view for describing a braiding angle of a first reinforcing body, and FIG. 5B is a view for describing a braiding angle of a second reinforcing body.

FIG. 6 is a schematic perspective view illustrating a distal end part of the catheter according to the first embodiment.

FIG. 7 is a schematic front view illustrating the distal end part of the catheter according to the first embodiment.

FIG. 8 is a plan view illustrating a state before a stylet is inserted into a catheter according to a second embodiment.

FIG. 9 is a side cross-sectional view illustrating the catheter according to the second embodiment.

FIG. 10 is a plan view illustrating a state after the stylet is inserted into the catheter according to the second embodiment.

### DETAILED DESCRIPTION OF PREFERRED EMBODIMENTS

Embodiments of the present invention are hereinafter described with reference to the accompanying drawings. The following description does not limit the technical scope or meaning of terms recited in claims. Dimensional ratios in the drawings are exaggerated for convenience of description, and might be different from actual ratios.

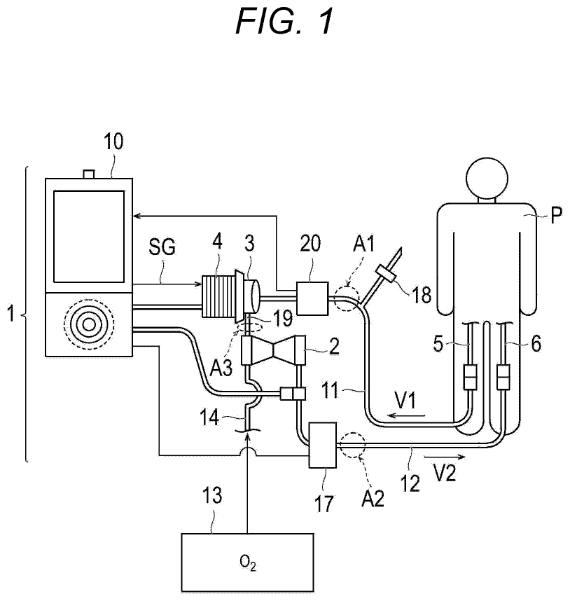

FIG. 1 is a system diagram illustrating an example of an extracorporeal circulation device to which a percutaneous catheter according to the embodiments of the present invention is applied, the extracorporeal circulation device used as percutaneous cardiopulmonary support (PCPS) that temporarily assists and substitutes for functions of the heart and lungs until a heart function recovers when the heart of a patient is weak.

According to an extracorporeal circulation device 1, it is possible to perform a veno-arterial (VA) procedure of removing blood from a vein (vena cava) of the patient by operating a pump, exchanging gas in the blood by an oxygenator to oxygenate the blood, and then returning the blood to an artery (aorta) of the patient again. The extracorporeal circulation device 1 is a device that assists the heart and lungs. Hereinafter, a procedure of removing the blood from the patient, performing predetermined extracorporeal treatment, and then supplying the blood into the body of the patient again is referred to as “extracorporeal circulation”.

As illustrated in FIG. 1, the extracorporeal circulation device 1 includes a circulation circuit that circulates the blood. The circulation circuit includes an oxygenator 2, a centrifugal pump 3, a drive motor 4 as a drive means for driving the centrifugal pump 3, a vein-side catheter (percutaneous catheter for blood removal) 5, an artery-side catheter (blood supply catheter) 6, and a controller 10 as a control unit.

The vein-side catheter (blood removal catheter) 5 is inserted from the femoral vein, and a distal end of the vein-side catheter 5 is indwelled in the right atrium via the inferior vena cava. The vein-side catheter 5 is connected to the centrifugal pump 3 via a blood removal tube (blood removal line) 11. The blood removal tube 11 is a pipeline that sends the blood.

The artery-side catheter (blood supply catheter) 6 is inserted from the femoral artery.

When the drive motor 4 operates the centrifugal pump 3 by a command SG of the controller 10, the centrifugal pump 3 may remove the blood from the blood removal tube 11 and pass the blood via the oxygenator 2, then return the blood to a patient P via a blood supply tube (blood supply catheter) 12.

The oxygenator 2 is arranged between the centrifugal pump 3 and the blood supply tube 12. The oxygenator 2 performs gas exchange (oxygenation and/or carbon dioxide removal) on the blood. The oxygenator 2 is, for example, a membrane oxygenator, and a hollow fiber membrane oxygenator is especially preferably used. Oxygen gas is supplied from an oxygen gas supply unit 13 to the oxygenator 2 through a tube 14. The blood supply tube 12 is a pipeline that connects the oxygenator 2 to the artery-side catheter 6.

As the blood removal tube 11 and the blood supply tube 12, for example, a pipeline made of an elastically deformable and flexible synthetic resin having high transparency such as a vinyl chloride resin or silicone rubber may be used. In the blood removal tube 11, the blood as liquid flows in a V1 direction, and in the blood supply tube 12, the blood flows in a V2 direction.

In the circulation circuit illustrated in FIG. 1, an ultrasonic bubble detection sensor 20 is arranged in the middle of the blood removal tube 11. A fast clamp 17 is arranged in the middle of the blood supply tube 12.

In a case where a bubble is mixed in the circulation circuit with the blood due to an erroneous operation of a three-way stopcock 18 or a breakage of the tube and the like during the extracorporeal circulation, then the ultrasonic bubble detection sensor 20 detects the mixed bubble. In a case where the ultrasonic bubble detection sensor 20 detects that there is the bubble in the blood sent in the blood removal tube 11, the ultrasonic bubble detection sensor 20 transmits a detection signal to the controller 10. On the basis of this detection signal, the controller 10 performs notification of a warning by an alarm, and decreases a rotation speed of the centrifugal pump 3 or stops the centrifugal pump 3. The controller 10 further instructs the fast clamp 17 to immediately close the blood supply tube 12 by the fast clamp 17. This prevents the bubble from being sent into the body of the patient P. The controller 10 controls an operation of the extracorporeal circulation device 1 to prevent mixing of the bubble into the body of the patient P.

A tube 11 (or tube 12, 19) of the circulation circuit of the extracorporeal circulation device 1 is provided with a pressure sensor. For example, the pressure sensor may be attached to any one or all of an attachment position A1 of the blood removal tube 11, an attachment position A2 of the blood supply tube 12 of the circulation circuit, and an attachment position A3 of a connection tube 19 that connects the centrifugal pump 3 to the oxygenator 2. As a result, a pressure in the tube 11 (12, 19) may be measured by the pressure sensor when the extracorporeal circulation is performed on the patient P by the extracorporeal circulation device 1. The attachment position of the pressure sensor is not limited to the attachment positions A1, A2, and A3 described above, and may be any position of the circulation circuit.

### First Embodiment

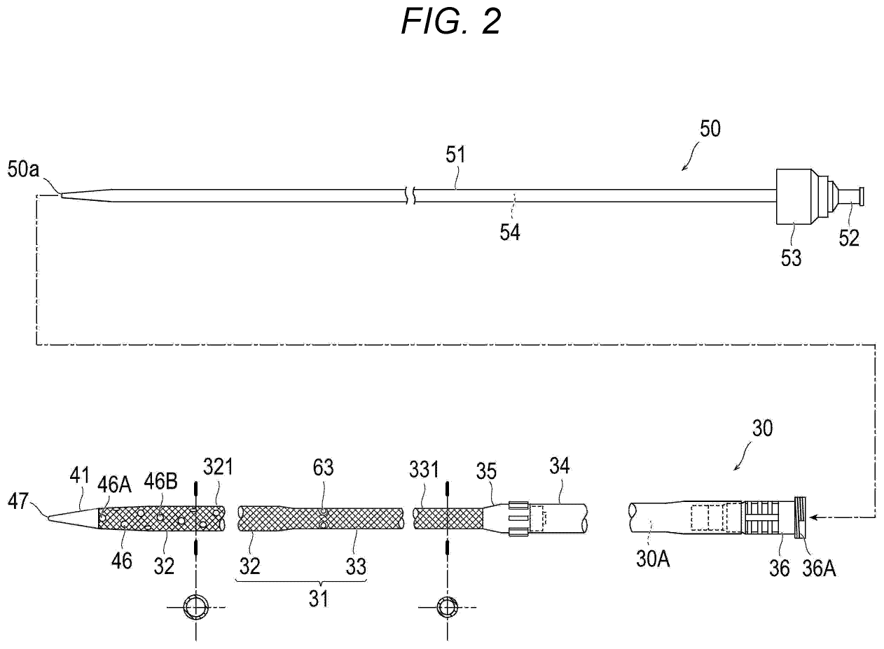

A percutaneous catheter (hereinafter, referred to as a “catheter”) 30 according to a first embodiment of the present invention is described with reference to FIGS. 2 to 6. FIGS. 2 to 6 are views for describing a configuration of the catheter 30 according to the first embodiment. The catheter 30 is used as the vein-side catheter (blood removal catheter) 5 in FIG. 1.

As illustrated in FIG. 2, the catheter 30 according to this embodiment includes a catheter tube (corresponding to the tube) 31 provided with a first side hole (or group or plurality of side holes) 63 and a second side hole (or group or plurality of side holes) 46, a distal end tip 41 arranged at a distal end of the catheter tube 31 and provided with a through-hole 47, a clamping tube 34 arranged on a proximal end side of the catheter tube 31, a catheter connector 35 that connects the catheter tube 31 to the clamping tube 34, and a lock connector 36.

In this specification, a side to be inserted into a living body is referred to as a “distal end” or a “distal end side”, and a hand side operated by an operator is referred to as a “proximal end” or a “proximal end side”. A distal end part means a certain range including the distal end (most distal end) and its periphery, and a proximal end part means a certain range including the proximal end (most proximal end) and its periphery.

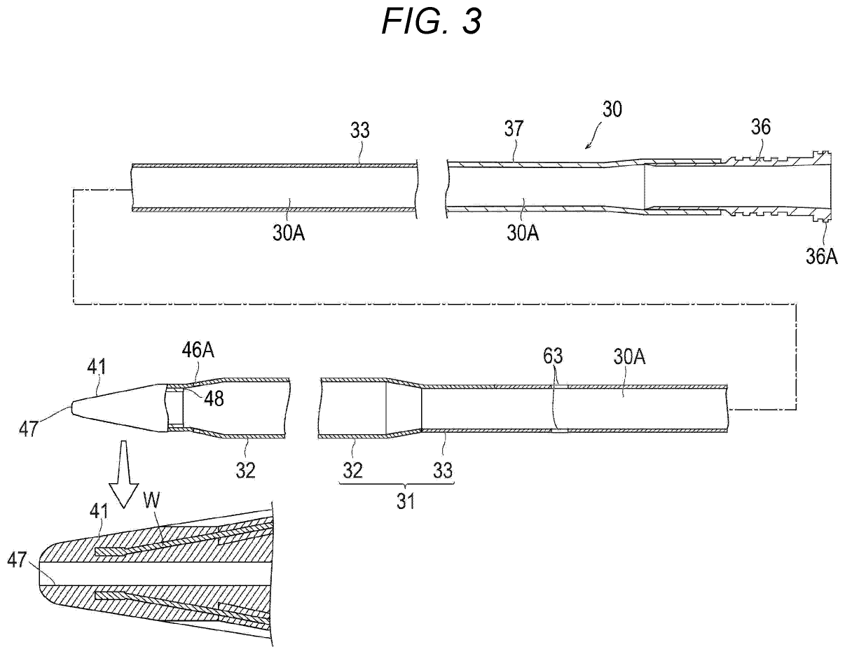

As illustrated in FIG. 3, the catheter 30 includes a lumen 30A penetrating from a distal end to a proximal end thereof. The through-hole 47 provided on the distal end tip 41, and the first and second side holes 63 and 46 provided on the catheter tube 31 are arranged in different blood removal targets in the living body such that the blood may be efficiently removed.

When the catheter 30 is inserted into the living body, a stylet 50 illustrated in FIG. 2 is used. The stylet 50 is inserted into the lumen 30A of the catheter 30, and the catheter 30 and the stylet 50 are inserted into the living body in a state of being integrated in advance. A method of using the catheter 30 is described later.

Hereinafter, each configuration of the catheter 30 is described.

As illustrated in FIG. 2, the catheter tube 31 includes a first tube 32 and a second tube 33 connected to a proximal end side of the first tube 32.

The first tube 32 is formed to have higher elasticity than that of the second tube 33. The first tube 32 is formed to have larger outer and inner diameters than those of the second tube 33.

Lengths of the first tube 32 and the second tube 33 are set to lengths necessary for arranging the through-hole 47 of the distal end tip 41 and the first and second side holes 63 and 46 of the catheter tube 31 in desired blood removal targets. The length of the first tube 32 may be set to, for example, 20 to 40 cm, and the length of the second tube 33 may be set to, for example, 20 to 30 cm.

In this embodiment, the blood removal targets are two sites: the right atrium and the inferior vena cava. The catheter 30 is inserted into the living body to be indwelled there such that the through-hole 47 of the distal end tip 41 and the second side hole 46 of the catheter tube 31 are arranged in the right atrium, and the first side hole 63 of the catheter tube 31 is arranged in the inferior vena cava.

In a state in which the through-hole 47, the second side hole 46, and the first side hole 63 are arranged in the blood removal targets, the first tube 32 is arranged in the inferior vena cava, which is a relatively large blood vessel, and the second tube 33 is arranged in the femoral vein, which is a relatively small blood vessel.

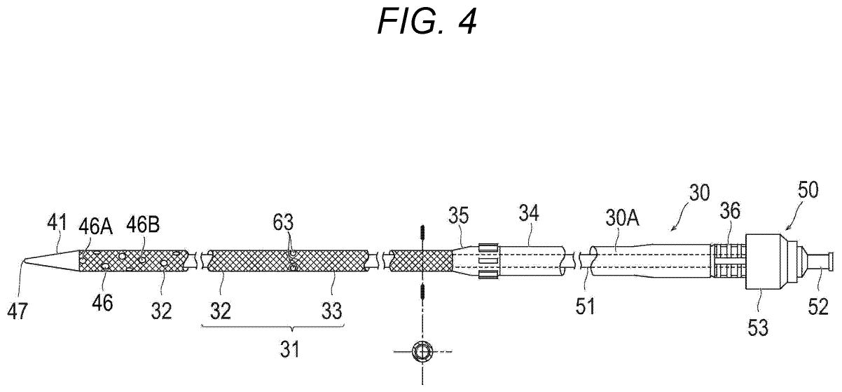

When the stylet 50 is inserted into the lumen 30A of the catheter 30, the first tube 32 having high elasticity extends in an axial direction and the outer and inner diameters thereof decrease as illustrated in FIG. 4. At that time, the outer diameter of the first tube 32 is substantially the same as the outer diameter of the second tube 33. Since the catheter 30 is inserted into the living body in a state in which the first tube 32 is extended in the axial direction and the outer and inner diameters thereof decrease, the catheter 30 may be inserted in a minimally invasive manner.

When the stylet 50 is removed from the lumen 30A of the catheter 30 after the catheter 30 is indwelled in the living body, the first tube 32 contracts from the state of being extended in the axial direction to have a larger inner diameter. Herein, the first tube 32 is arranged in the inferior vena cava, which is a relatively large blood vessel. Therefore, the outer diameter of the first tube 32 may be increased, and accordingly, the inner diameter may be increased.

Herein, a pressure loss in the first tube 32 is obtained by a total length of the first tube 32×(average) passage sectional area. That is, by increasing the inner diameter of the first tube 32, the pressure loss in the first tube 32 is decreased. When the pressure loss in the first tube 32 is decreased, a flow rate of the blood flowing through the circulation circuit increases. Therefore, in order to obtain a sufficient blood circulation amount, it is necessary to increase the inner diameter of the first tube 32.

In contrast, in a case where a wall thickness is substantially constant, when the inner diameters of the first tube 32 and the second tube 33 are increased, the outer diameters increase, so that a burden on the patient increases when the catheter 30 is inserted into the living body, which hinders a minimally invasive procedure.

From the above-described viewpoint, the inner diameter of the first tube 32 may be set to, for example, 9 to 11 mm, and the inner diameter of the second tube 33 may be set to, for example, 4 to 8 mm. The wall thickness of each of the first tube 32 and the second tube 33 may be set to, for example, 0.4 to 0.5 mm.

As illustrated in FIG. 2, it is preferable that a distal end part of the first tube 32 forms a tapered portion that gradually becomes thinner from the center of the first tube 32 outward in the axial direction. As a result, the inner diameter of a distal end of the first tube 32 is continuous to an inner diameter of the distal end tip 41 arranged on a distal end side thereof.

Hereinafter, the configurations of the first tube 32 and the second tube 33 are described in further detail.

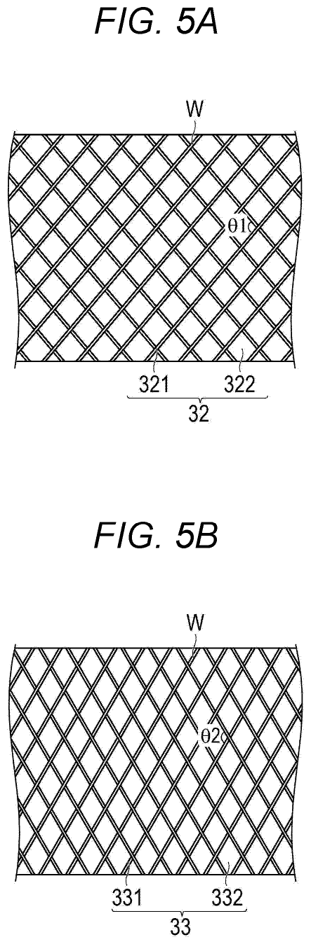

As illustrated in FIG. 5A, the first tube 32 includes a first reinforcing body 321 including wires W braided so as to cross each other, and a first resin layer 322 provided so as to cover the first reinforcing body 321.

As illustrated in FIG. 5B, the second tube 33 includes a second reinforcing body 331 including wires W braided so as to cross each other, and a second resin layer 332 provided so as to cover the second reinforcing body 331.

As illustrated in FIG. 5A, the first reinforcing body 321 is formed by braiding the wires W to form a braiding angle θ1. As illustrated in FIG. 5B, the second reinforcing body 331 is formed by braiding the wires W to form a braiding angle θ2.

In this specification, the braiding angles θ1 and θ2 are defined as inner angles in the axial direction out of the angles formed by the crossing wires W, as illustrated in FIGS. 5A and 5B.

As illustrated in FIGS. 5A and 5B, the braiding angle θ1 of the first reinforcing body 321 is made smaller than the braiding angle θ2 of the second reinforcing body 331. Therefore, an inclination angle of the wire W forming the first reinforcing body 321 with respect to the axial direction is smaller than that in a case where the braiding angle of the first reinforcing body 321 is larger than the braiding angle of the second reinforcing body 331.

Herein, as the first tube 32 extends in the axial direction, the wire W forming the first reinforcing body 321 of the first tube 32 is deformed such that the inclination angle with respect to the axial direction gradually decreases. When the inclination angle of the wire W forming the first reinforcing body 321 of the first tube 32 with respect to the axial direction becomes approximately 0, the extension of the first tube 32 in the axial direction is restricted.

Therefore, by making the braiding angle θ1 of the first reinforcing body 321 smaller than the braiding angle θ2 of the second reinforcing body 331, as compared with a case where the braiding angle of the first reinforcing body 321 is larger than the braiding angle of the second reinforcing body 331, an extension distance in the axial direction of the first tube 32 accompanying the insertion of the stylet 50 into the catheter 30 is shortened.

The braiding angle θ1 of the first reinforcing body 321 is not especially limited, but is 100 to 120 degrees. The braiding angle θ2 of the second reinforcing body 331 is not especially limited, but is 130 to 150 degrees. By making the braiding angle θ2 of the second reinforcing body 331 larger than the braiding angle θ1 of the first reinforcing body 321, kink resistance of the second reinforcing body 331 may be improved. Therefore, the catheter 30 may be suitably inserted into the living body in the femoral vein having a complicated configuration.

As illustrated in FIGS. 5A and 5B, the first reinforcing body 321 of the first tube 32 is braided sparser than the second reinforcing body 331 of the second tube 33. According to this configuration, the first tube 32 may be made softer than the second tube 33, and elasticity may be enhanced.

In this embodiment, the wire W is formed of a well-known shape memory material of shape memory metal or a shape memory resin. As the shape memory metal, for example, a titanium-based (Ni—Ti, Ti—Pd, Ti—Nb—Sn and the like) or copper-based alloy may be used. As the shape memory resin, for example, an acrylic resin, a transisoprene polymer, polynorbornene, a styrene-butadiene copolymer, and polyurethane may be used.

Since the wire W is formed of the shape memory material, a contraction distance in the axial direction of the first tube 32 accompanying the removal of the stylet 50 from the catheter 30 is the same as the extension distance in the axial direction of the first tube 32 accompanying the insertion of the stylet 50 into the catheter 30.

A diameter of the wire W is preferably 0.1 to 0.2 mm.

By setting the diameter of the wire W to 0.1 mm or larger, a function as the reinforcing body for improving strength may be suitably exhibited.

In contrast, by setting the diameter of the wire W to 0.2 mm or smaller, the inner diameter of the first tube 32 may be increased while decreasing the outer diameter thereof, so that it is possible to achieve both suppression in burden on the body of the patient at the time of insertion of the catheter 30 and decrease in pressure loss. At that time, the wire W may be prevented from being exposed from the first resin layer 322 even in a site where the wire W is braided into two layers. In this embodiment, a cross section of the wire W is circular, but is not limited thereto, and may be rectangular, square, elliptical and the like.

The first resin layer 322 of the first tube 32 is formed of a soft material having hardness lower than that of the second resin layer 332 of the second tube 33. According to this configuration, the first tube 32 may be made softer than the second tube 33, and elasticity may be enhanced.

The first and second resin layers 322 and 332 may be formed using vinyl chloride, silicon, polyethylene, nylon, urethane, polyurethane, a fluororesin, a thermoplastic elastomer resin and the like, or a composite material thereof.

The silicon material has high biocompatibility, and the material itself is soft, so that this has an advantage that this does not easily damage a blood vessel. The polyethylene material is soft and has hardness to withstand a pressure. Moreover, the polyethylene material has biocompatibility comparable to that of the silicon material. The polyethylene material is harder than silicon, and has an advantage that this is easily inserted into a thin blood vessel. The polyurethane material has an advantage that this becomes soft after insertion. As the materials of the first and second resin layers 322 and 332, applicable materials may be used by taking advantage of the features of these materials.

A hydrophilic coating may be applied to the polyurethane material. In this case, since a tube surface is smooth, this may be easily inserted into the blood vessel, and the blood vessel wall is less likely to be damaged. The blood and proteins are less likely to adhere, and it may be expected that thrombus formation is prevented.

A method of forming the tubes 32 and 33 is not especially limited, but they may be formed by, for example, dip coating (immersion method), insert molding and the like. It is sufficient that at least outer surfaces of the reinforcing bodies 321 and 331 are covered with the resin layers 322 and 332, respectively.

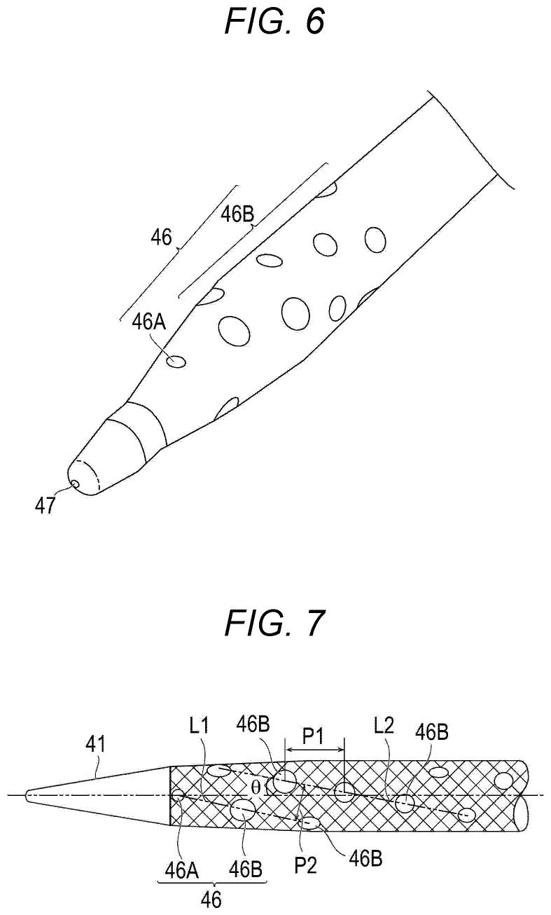

As illustrated in FIG. 2, the first tube 32 includes the second side hole 46. As illustrated in FIGS. 6 and 7, the second side hole 46 includes a hole portion 46A provided on the distal end side and a third side hole (corresponding to a side hole) 46B provided on a proximal end side of the hole portion 46A. The second side hole 46 serves as a blood removal hole.

The hole portion 46A is a “tip side hole” provided on a distal end side of the third side hole 46B. As illustrated in FIG. 3, the hole portion 46A is formed in the vicinity of a receiving surface 48 to be described later. As illustrated in FIG. 6, an inner diameter of the hole portion 46A is made smaller than an inner diameter of the third side hole 46B. Since the hole portion 46A is formed in the vicinity of the receiving surface 48 in this manner, the blood flowing into the lumen 30A via the hole portion 46A may decrease stagnation of the blood in the vicinity of the receiving surface 48, and suitably suppress occurrence of thrombus. The hole portion 46A is a substantially circular hole, and a diameter thereof may be set to, for example, 1.0 to 2.5 mm.

As illustrated in FIGS. 6 and 7, a plurality of third side holes 46B is formed spirally in the axial direction. As illustrated in FIG. 7, the third side holes 46B are formed to be inclined at an angle θ with respect to the axial direction. The angle θ is not especially limited, but is 30 to 45 degrees, for example.

Specifically, the plurality of third side holes 46B is arranged along a plurality of virtual lines L1 and L2. Thus, one third side hole 46B and another third side hole 46B adjacent to the one third side hole 46B arranged on one virtual line are arranged at different positions in the axial direction and circumferential direction of the catheter tube 31. Therefore, it is possible to suppress collision in the blood vessel between the blood sent through one third side hole 46B and the blood sent through another third side hole 46B. Therefore, in the catheter 30 according to this embodiment, it is possible to suitably suppress the blood from being stagnated in the blood vessel due to collision of the blood flowing in via the third side holes 46B, and to stably remove the blood.

An interval P1 (refer to FIG. 7) between one third side hole 46B and another third side hole 46B in the axial direction may be set to, for example, 3.0 to 5.0 mm. The number, the inner diameter, and the interval P1 of the third side holes 46B are not limited thereto, and may be appropriately set as necessary. The shape of the third side hole 46B is not limited to a substantially circular shape, and may be a substantially elliptical shape, for example.

The virtual lines L1 and L2 (refer to FIG. 7) are parallel to each other, and an interval P2 between the virtual lines L1 and L2 may be appropriately adjusted. The third side holes 46B arranged on the virtual line L2 may be arranged so as to be located between two adjacent third side holes 46B out of the third side holes 46B arranged on the virtual line L1 in the axial direction of the catheter tube 31.

In this embodiment, it is described that the number of virtual lines on which the plurality of third side holes 46B is arranged is two, but this is not especially limited. The interval between one virtual line and the adjacent virtual line is not especially limited, and may be appropriately set as necessary.

As illustrated in FIGS. 6 and 7, the third side holes 46B are formed to have a smaller inner diameters toward the proximal end side. According to the catheter 30 formed in this manner, since the third side hole 46B on the proximal end side is formed to have a smaller diameter than that of the third side hole 46B on the distal end side, a blood removal amount in the third side hole 46B on the distal end side may be made larger than the blood removal amount from the third side hole 46B on the proximal end side.

As illustrated in FIG. 2, the second tube 33 includes the first side hole 63. The first side hole 63 serves as a blood removal hole. A plurality of first side holes 63 is preferably provided in the circumferential direction. In this embodiment, the second tube 33 is provided with four first side holes 63 in the circumferential direction. As a result, even if one first side hole 63 is adsorbed to the blood vessel wall and blocked by the blood removal, the blood removal may be performed by another first side hole 63, so that blood circulation may be stably performed.

As illustrated in FIGS. 2 to 4, the distal end tip 41 is arranged at the distal end of the first tube 32. The distal end tip 41 has a tapered shape a diameter of which is gradually decreased toward the distal end side.

A flat receiving surface 48 that abuts a flat surface 50a of the stylet 50 used before the catheter 30 is inserted into the living body is formed inside the distal end tip 41.

As illustrated in FIG. 3, the distal end tip 41 is formed to house a distal end of the wire W. The distal end tip 41 includes the through-hole 47. The through-hole 47 serves as a blood removal hole. The through-hole 47 of the distal end tip 41 forms a part of the lumen 30A of the catheter 30. The distal end tip 41 may be formed of, for example, urethane.

By fixing a hard distal end tip 41 to the distal end part of the first tube 32, it is possible to effectively prevent the first tube 32 from being crushed at the time of blood removal.

As illustrated in FIGS. 2 to 4, the clamping tube 34 is provided on a proximal end side of the second tube 33. A lumen into which the stylet 50 may be inserted is provided inside the clamping tube 34. The clamping tube 34 may be formed using a material similar to that of the catheter tube 31.

As illustrated in FIGS. 2 and 4, the catheter connector 35 connects the second tube 33 to the clamping tube 34. A lumen into which the stylet 50 may be inserted is provided inside the catheter connector 35.

As illustrated in FIGS. 2 to 4, the lock connector 36 is connected to a proximal end side of the clamping tube 34. A lumen into which the stylet 50 may be inserted is provided inside the lock connector 36. A male screw portion 36A provided with a screw thread is provided on an outer surface on a proximal end side of the lock connector 36.

Next, a configuration of the stylet 50 is described.

As illustrated in FIG. 2, the stylet 50 includes a stylet tube 51 provided so as to extend in the axial direction, a stylet hub 52 to which a proximal end of the stylet tube 51 is fixed, and a screw ring 53 provided at a distal end of the stylet hub 52.

The stylet tube 51 is an elongated body extending in the axial direction and having relatively high rigidity. An entire length in the axial direction of the stylet tube 51 is made longer than an entire length in the axial direction of the catheter 30. The stylet tube 51 is provided with a guidewire lumen 54 into which a guidewire (not illustrated) may be inserted. The stylet tube 51 is guided by the guide wire to be inserted into the living body together with the catheter 30. After the catheter 30 is indwelled in the living body, the stylet tube 51 is removed from the catheter 30 by pulling out the stylet hub 52 to the proximal end side.

As illustrated in FIG. 2, a distal end of the stylet tube 51 is provided with the flat surface 50a that the receiving surface 48 of the distal end tip 41 abuts. The stylet tube 51 has relatively high rigidity and has stiffness that enables transmission of a pushing force to the distal end side by a hand side operation to the distal end tip 41. Therefore, the stylet tube 51 plays a role of expanding a narrow blood vessel by allowing the flat surface 50a thereof to abut the receiving surface 48 of the distal end tip 41 and pushing the distal end tip 41 toward the distal end side.

The screw ring 53 includes a female screw portion (not illustrated) provided with a screw groove on an inner surface of an inner cavity. The stylet 50 may be attached to the catheter 30 by screwing the female screw portion of the screw ring 53 into the male screw portion 36A of the lock connector 36.

<Method of Using Catheter>

Next, a method of using the above-described catheter 30 is described. FIG. 2 illustrates a state before the stylet tube 51 of the stylet 50 is inserted into the lumen 30A of the catheter 30, and FIG. 4 illustrates a state after the stylet tube 51 is inserted into the lumen 30A of the catheter 30.

First, as illustrated in FIG. 4, the stylet tube 51 of the stylet 50 is inserted into the lumen 30A of the catheter 30. The stylet tube 51 sequentially passes through the inside of the second tube 33 and the first tube 32, and the flat surface 50a of the stylet tube 51 abuts the receiving surface 48 of the distal end tip 41.

Herein, an entire length in the axial direction of the stylet tube 51 is made longer than an entire length in the axial direction of the catheter 30 as illustrated in FIG. 2. Therefore, the distal end tip 41 is pressed toward the distal end side in a state in which the flat surface 50a of the stylet tube 51 abuts the receiving surface 48 of the distal end tip 41. As a result, the distal end of the first tube 32 fixed to the distal end tip 41 is pulled toward the distal end side. As a result, the catheter 30 receives a force to extend in the axial direction, and the first tube 32 having relatively high elasticity out of the catheter 30 extends in the axial direction. Thereafter, the proximal end of the catheter 30 is fixed to the stylet hub 52.

Next, the catheter 30 into which the stylet 50 is inserted is inserted along the guide wire (not illustrated) inserted in advance into a target site in the living body. At that time, since the stylet 50 is inserted into the catheter 30, the outer diameter of the first tube 32 is substantially the same as the outer diameter of the second tube 33, and the catheter 30 may be inserted into the living body in a minimally invasive manner, and the burden on the body of the patient may be suppressed.

The catheter 30 is inserted into the living body until the through-hole 47 of the distal end tip 41 and the second side hole 46 of the catheter tube 31 are arranged in the right atrium, and the first side hole 63 of the catheter tube 31 is arranged in the inferior vena cava to be indwelled there. In a state in which the through-hole 47, the first side hole 63, and the second side hole 46 are arranged in the blood removal targets, the first tube 32 is arranged in the inferior vena cava, which is a relatively large blood vessel, and the second tube 33 is arranged in the femoral vein, which is a relatively small blood vessel.

Next, the stylet tube 51 and the guide wire are removed from the catheter 30. At that time, the stylet tube 51 and the guide wire are temporarily pulled out to a site of the clamping tube 34 of the catheter 30 and clamped by forceps (not illustrated), and then completely removed from the catheter 30. When the stylet tube 51 is removed from the lumen of the catheter 30, the catheter 30 is released from the force to axially extend that the catheter 30 receives from the stylet 50. Therefore, the first tube 32 contracts in the axial direction, and the inner diameter of the first tube 32 increases. As a result, the pressure loss in the first tube 32 may be decreased, and a required flow rate of liquid may be secured.

Next, the lock connector 36 of the catheter 30 is connected to the blood removal tube 11 of the extracorporeal circulation device in FIG. 1. After confirming that the connection of the catheter on the blood supply side is completed, the forceps of the clamping tube 34 are released to start the extracorporeal circulation.

When the extracorporeal circulation ends, the catheter 30 is removed from the blood vessel and hemostatic repair is performed by a surgical procedure as necessary at an insertion site.

As described above, the catheter 30 according to this embodiment is the catheter 30 provided with the lumen 30A through which the blood passes. The catheter 30 includes the catheter tube 31 extending in the axial direction and the third side hole 46B provided on a distal end part of the catheter tube 31 and communicating the lumen 30A with the outside of the catheter tube 31. A plurality of third side holes 46B is spirally arranged in the axial direction of the catheter tube 31. Out of the third side holes 46B, the third side hole 46B on the proximal end side is formed to have the diameter smaller than that of the third side hole 46B on the distal end side.

According to the catheter 30 formed as described above, since the third side holes 46B are formed spirally in the axial direction, it is possible to suitably suppress collision of the blood flowing in via the third side holes 46B. Therefore, deterioration in blood removal efficiency may be suitably suppressed.

According to the catheter 30 formed in the above-described manner, since the third side hole 46B on the proximal end side is formed to have the diameter smaller than that of the third side hole 46B on the distal end side, the blood removal amount in the third side hole 46B on the distal end side may be made larger than the blood removal amount from the third side hole 46B on the proximal end side.

From above, according to the catheter 30 formed as described above, even in a case where a plurality of third side holes 46B is provided, it is possible to suitably suppress the deterioration in blood removal efficiency and to make the blood removal amount in the third side hole 46B on the distal end side larger than the blood removal amount from the third side hole 46B on the proximal end side.

The catheter 30 further includes the hole portion 46A provided on the distal end side of the third side hole 46B and having the diameter smaller than that of the third side hole 46B. According to the catheter 30 formed in this manner, the blood flowing into the lumen 30A via the hole portion 46A may decrease stagnation of the blood in the vicinity of the receiving surface 48, and suitably suppress occurrence of thrombus.

### Second Embodiment

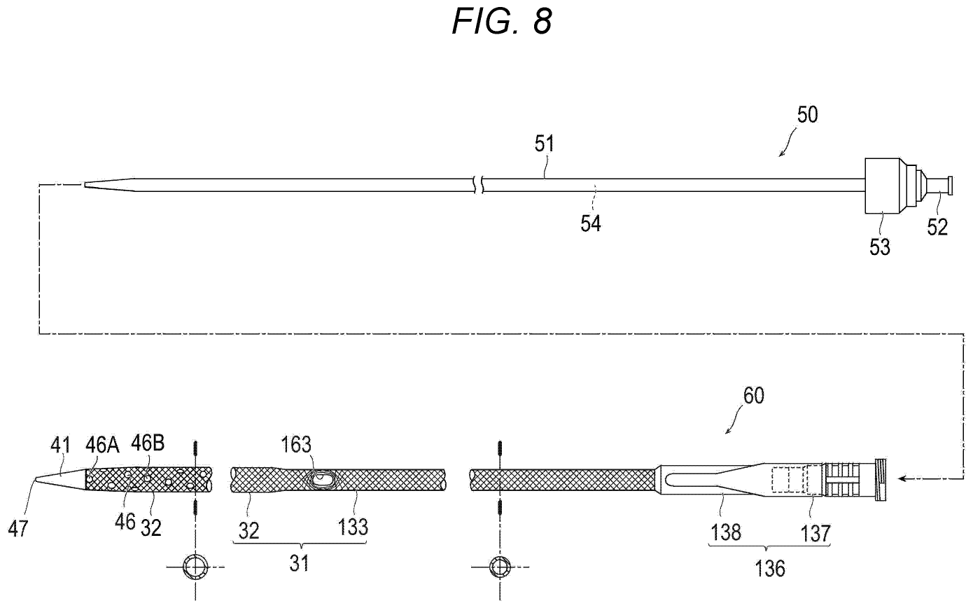

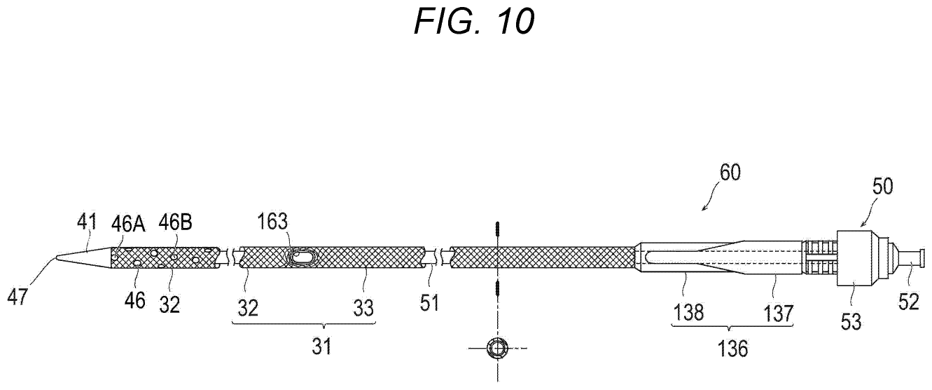

A percutaneous catheter (hereinafter, referred to as a “catheter”) 60 according to a second embodiment of the present invention is described with reference to FIGS. 8 to 10. FIGS. 10 to 12 are views for describing a configuration of the catheter 60 according to the second embodiment.

The catheter 60 is a so-called double lumen catheter, and may simultaneously perform both blood supply and blood removal. Therefore, in this embodiment, a procedure is performed using only one catheter 60 without using two catheters of a vein-side catheter (blood removal catheter) 5 and an artery-side catheter (blood supply catheter) 6 in the extracorporeal circulation device in FIG. 1.

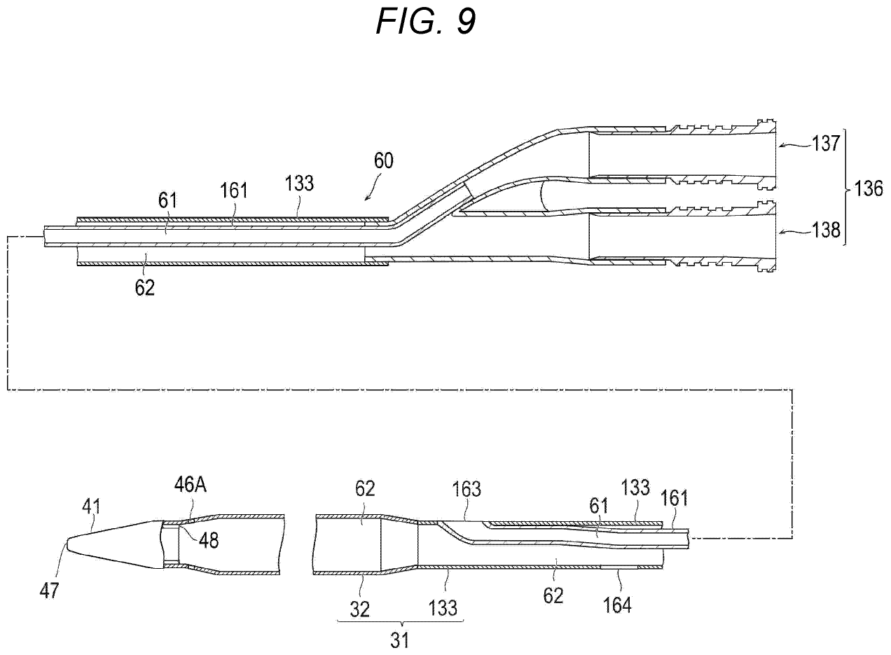

As illustrated in FIGS. 8 and 9, the catheter 60 according to this embodiment is different from the catheter 30 according to the first embodiment in having a double tube structure in which a third tube 161 provided with a first lumen 61 communicating with a blood supply side hole 163 is arranged in an inner cavity of a second tube 133.

According to the catheter 60, it is possible to perform veno-venous (VV) oxygenator extracorporeal blood circulation of removing blood from a vein (vena cava) of a patient by operating a pump of the extracorporeal circulation device, exchanging gas in the blood by an oxygenator to oxygenate the blood, and then returning the blood to an artery (aorta) of the patient again.

Hereinafter, each configuration of the catheter 60 is described. Parts common to those of the first embodiment are not described, and parts having features only in the second embodiment are described. The same portions as those of the first embodiment described above are assigned with the same reference numerals, and redundant description is omitted.

As illustrated in FIGS. 8 to 10, the catheter 60 includes a first tube 32, the second tube 133, a distal end tip 41 arranged at a distal end of the first tube 32, and the third tube 161 arranged in the inner cavity of the second tube 133. Since configurations of the first tube 32 and the distal end tip 41 are the same as those of the catheter 30 of the first embodiment, the description thereof is omitted.

As illustrated in FIG. 9, the catheter 60 includes the first lumen 61 serving as a blood supply path and a second lumen 62 serving as a blood removal path.

The first lumen 61 is formed in an inner cavity of the third tube 161. The second lumen 62 is formed in the inner cavities of the first tube 32 and the second tube 133, and penetrates from a distal end to a proximal end.

The second tube 133 is provided with the blood supply side hole 163 communicating with the first lumen 61, which is the blood supply path.

The second tube 133 is provided with a blood removal side hole 164 communicating with the second lumen 62, which is a blood removal path.

The blood supply side hole 163 and the blood removal side hole 164 are formed into an elliptical shape.

The third tube 161 is inserted into the second lumen 62 from a proximal end side of the second tube 133 and connected to the blood supply side hole 163.

The blood supply side hole 163 is arranged in a blood supply target in a living body, and the blood oxygenated by the oxygenator is sent into the living body via the blood supply side hole 163.

A through-hole 47 provided on the distal end tip 41, a second side hole 46 provided on the first tube 32, and the blood removal side hole 164 provided on the second tube 133 are arranged in different blood removal targets in the living body such that the blood may be efficiently removed. Even when the through-hole 47, the second side hole 46, or the blood removal side hole 164 is adsorbed to a blood vessel wall and blocked, the blood removal may be performed by a hole not blocked, so that extracorporeal circulation may be stably performed.

In this embodiment, the catheter 60 is inserted from the internal jugular vein of the neck, and a distal end thereof is indwelled in the inferior vena cava via the superior vena cava and the right atrium. The blood supply target is the right atrium and the blood removal target are two sites: the superior vena cava and the inferior vena cava.

The catheter 60 is inserted into the living body to be indwelled there such that the through-hole 47 of the distal end tip 41 and the second side hole 46 of the first tube 32 are arranged in the inferior vena cava, and the blood removal side hole 164 of the second tube 133 is arranged in the internal jugular vein in a state in which the stylet 50 is inserted as illustrated in FIG. 12.

As in the first embodiment, the first tube 32 is formed to have a larger inner diameter than that of the second tube 133. In a state in which the through-hole 47, the second side hole 46, and the blood removal side hole 164 are arranged in the blood removal targets, the first tube 32 is arranged in the inferior vena cava, which is a relatively large blood vessel, and the second tube 133 is arranged in the femoral vein, which is a relatively small blood vessel.

As illustrated in FIG. 9, a lock connector 136 includes a first lock connector 137 communicating with the first lumen 61 and a second lock connector 138 provided in parallel with the first lock connector 137 and communicating with the second lumen 62. The lock connector 136 is a Y-shaped Y connector formed by branching the first lock connector 137 from the second lock connector 138.

The first lock connector 137 is connected to a proximal end part of the third tube 161. The second lock connector 138 is coaxially connected to a proximal end part of the second tube 133. A blood supply tube (blood supply line) is connected to the first lock connector 137, and a blood removal tube (blood removal line) is connected to the second lock connector 138.

The first tube 32 exhibits the same function as that of the first embodiment, and has the same effect.

As described above, according to the catheter 60 according to this embodiment, it is possible to achieve both functions of blood removal and blood supply with one catheter.

Although the catheter according to the present invention is described above through the embodiments, the present invention is not limited to only the configuration described in the embodiments and variations, and may be appropriately changed on the basis of the recitation in claims.

For example, in the first embodiment described above, the catheter 30 includes the hole portion 46A having the diameter smaller than that of the third side hole 46B provided on the distal end side of the third side hole 46B; however, it is also possible that the catheter is not provided with the hole portion.

The material forming the wire W is not limited to the shape memory material as long as the material has a restoring force to deform and return to its original shape and has a function of reinforcing the resin layer; for example, this may be a known elastic material.

What is claimed is:

1. A percutaneous catheter provided with a lumen through which blood passes, the percutaneous catheter comprising:
a tube extending in an axial direction; and
a side hole provided on a distal end part of the tube and communicating the lumen with an outside of the tube, wherein
a plurality of side holes is spirally arranged in the axial direction of the tube, and
the side hole on a proximal end side out of the side holes is formed to have a diameter smaller than a diameter of the side hole on a distal end side.

2. The percutaneous catheter according to claim 1, further comprising:
a hole portion provided on a distal end side of the side hole and having a diameter smaller than the diameter of the side hole.

3. The percutaneous catheter according to claim 1, wherein an angle of the spirally formed side holes with respect to the axial direction in plan view is 30 to 45 degrees.

4. A percutaneous catheter provided with a lumen through which blood passes which is removed by the percutaneous catheter from a living body, the percutaneous catheter comprising:
a tube extending in an axial direction between a distal end and a proximal end; and
a distal tip;
wherein the tube includes a plurality of distal side holes provided on a distal end part of the tube and communicating the lumen with an outside of the tube configured to remove the blood from a first target in the living body, wherein the plurality of distal side holes is spirally arranged in the axial direction of the tube along a virtual line from a proximal end side to a distal end side; and
wherein one of the plurality of distal side holes at the proximal end side is formed to have a diameter smaller than a diameter of another one of the plurality of distal side holes at the distal end side, whereby a blood removal amount of the distal side holes at the distal end side is larger than a blood removal amount of the distal side holes at the proximal end side.

5. The percutaneous catheter according to claim 4, wherein a receiving surface is provided at the distal tip for receiving an end of a stylet which is inserted into the percutaneous catheter for placement in the living body, wherein the percutaneous catheter further comprises:
a tip side hole provided through the tube between the distal tip and the distal end side, wherein the tip side hole has a diameter smaller than the diameter of the another one of the plurality of distal side holes at the distal end side.

6. The percutaneous catheter according to claim 4, wherein an angle of the spirally formed side holes with respect to the axial direction in plan view is 30 to 45 degrees.

7. A percutaneous catheter provided with a lumen through which blood passes which is removed by the percutaneous catheter from a living body, the percutaneous catheter comprising:
a tube extending in an axial direction between a distal end and a proximal end; and
a distal tip;
wherein the tube includes a plurality of distal side holes provided on a distal end part of the tube and communicating the lumen with an outside of the tube configured to remove the blood from a first target in the living body, wherein the plurality of distal side holes is spirally arranged in the axial direction of the tube along a virtual line from a proximal end side to a distal end side;
wherein one of the plurality of distal side holes at the proximal end side is formed to have a diameter smaller than a diameter of another one of the plurality of distal side holes at the distal end side, whereby a blood removal amount of the distal side holes at the distal end side is larger than a blood removal amount of the distal side holes at the proximal end side; and
wherein the tube includes a plurality of intermediate side holes provided on the tube proximally from the plurality of distal side holes, wherein the intermediate side holes communicate the lumen with an outside of the tube and are configured to remove the blood from a second target in the living body.

8. The percutaneous catheter according to claim 7, wherein a receiving surface is provided at the distal tip for receiving an end of a stylet which is inserted into the percutaneous catheter for placement in the living body, wherein the percutaneous catheter further comprises:
a tip side hole provided through the tube between the distal tip and the distal end side, wherein the tip side hole has a diameter smaller than the diameter of the another one of the plurality of distal side holes at the distal end side.

9. The percutaneous catheter according to claim 7, wherein an angle of the spirally formed side holes with respect to the axial direction in plan view is 30 to 45 degrees.
